(19) United States
(12) Patent Application Publication
(10) Pub. No.: US 2004/0116691 A1
(43) Pub. Date: Jun. 17, 2004

(54) Process for cyclizing hydrolysis of an aminonitrile compound to a lactam

(76) Inventors: Cecile Rosier, Soucieu En Jarrest (FR); Aline Seigneurin, Le Chesnay (FR)

Correspondence Address: BURNS DOANE SWECKER & MATHIS L L P, POST OFFICE BOX 1404, ALEXANDRIA, VA, 22313-1404, US

(21) Appl. No.: 10/472,473

(22) PCT Filed: Mar. 19, 2002
(86) PCT No.: PCT/FR02/00957

(30) Foreign Application Priority Data
Mar. 21, 2001 (FR) 0103826

(51) Int. Cl. C07D 201/02

(52) U.S. Cl. 540/539

(57) ABSTRACT

The invention pertains to a process for cyclizing hydrolysis of an aminonitrile compound to a lactam in the presence of a catalyst.

It relates more particularly to a process for cyclizing hydrolysis of an aminonitrile compound in the presence of a solid catalyst of clay type. The invention applies particularly to the preparation of ε-caprolactam by cyclizing hydrolysis of aminocapronitrile.

The present invention pertains to a process for cyclizing hydrolysis of an aminonitrile compound to a lactam in the presence of a catalyst.

It relates more particularly to a process for cyclizing hydrolysis of an aminonitrile compound to a lactam in the presence of a solid catalyst of clay type.

Lactams, such as epsilon-caprolactam, are starting compounds for the production of polyamides and especially PA 6.

One of the various known processes for synthesizing these lactams is the cyclizing hydrolysis of the corresponding aminonitrile, and more particularly of a corresponding aliphatic aminonitrile, in the presence of water and a catalyst.

Thus U.S. Pat. No. 2,357,484 describes a vapour phase lactam preparation process using as its catalyst activated alumina, silica in gel form or borophosphoric acid.

U.S. Pat. No. 4,628,085 likewise describes a process for preparing lactams by cyclizing hydrolysis in vapour phase, in the presence of a silicon-based catalyst with a specific surface of more than-250 m²/g. This reaction is conducted in the presence of hydrogen and ammonia.

Application WO 98/0669 proposes catalysts based on hydrated or unhydrated metal oxides, the metals being selected from the group consisting of tin, zirconium, hafnium, bismuth, vanadium, niobium, and tantalum or mixtures thereof. The cycle time of these catalysts is very short and is not compatible with an industrial exploitation of the lactam production process.

Application wo 96/22974 proposes a process for cyclizing hydrolysis of aminonitrile in vapour phase which uses as its catalyst an alumina which exhibits regions of particular specific surface and pore volume.

Metal phosphates and zeolites are also known catalysts of the reaction for preparing lactams by cyclizing hydrolysis.

In general, the catalysts used in the prior art processes exhibit good initial activity and provide good selectivity of the reaction for converting aminonitrile into lactam. On the other hand, it is often found that they are rapidly deactivated, which is a very great drawback for industrial implementation of the said processes.

Moreover, the process according to U.S. Pat. No. 4,628,085 employs a highly complex reaction mixture, necessitating operations for separation and recycling at the end of reaction, which greatly complicate the said process.

In the selection of an appropriate catalyst for the cyclizing hydrolysis of an aminonitrile to a lactam, it is advantageous to have the best possible balance between cost, catalyst activity and selectivity, catalyst life, simplicity of catalyst employment, etc.

The present invention proposes novel catalysts of clay type which satisfactorily meet the various criteria mentioned above.

With this aim, the invention provides a process for cyclizing hydrolysis of an aminonitrile compound to a lactam by reaction of an aminonitrile of general formula (I) below:

N≡C—R—NH₂ (I)

in which:

R represents a substituted or unsubstituted, linear or branched, saturated or unsaturated aliphatic, cycloaliphatic or arylaliphatic radical containing from 1 to 12 carbon atoms with water in the presence of a solid catalyst, characterized in that the catalyst is a clay.

Clays are phyllosilicates which are classified into groups according to their nature and their physicochemical properties; among these groups mention may be made of kaolins, serpentines, smectites or montmorillonites, illites or micas, glauconites, chlorites or vermiculites, attapulgites or sepiolites, mixed-layer clays, allophanes or imogolites, and clays with a high alumina content.

Some clays possess a lamellar structure with an expandable network. They have the particular feature of adsorbing diverse solvents, particularly water, between their constituent sheets, thereby giving rise to swelling of the solid following weakening of the electrostatic bonds between the sheets. These clays belong essentially to the smectite group (or montmorillonite group) and some of them to the vermiculite group.

Their structure is composed of “elementary” sheets having three layers: two single layers of SiO₄ tetrahedra in which some of the silicon may be replaced by other cations in the tetrahedral position, such as Al³⁺ or possibly of Fe³⁺, and, between these two tetrahedra layers, a layer of oxygen octahedra at the centre of which are located metal-cations such as Al³⁺, Fe³ and Mg²⁺. This octahedral layer consists of a compact stack of oxygens originating either from the apices of the aforementioned tetrahedra or from hydroxyl groups, OH. The compact hexagonal network of these oxygens contains 6 octahedral cavities.

When the metal cations occupy 4 of these cavities (2 cavities out of 3 as in the case of aluminium, for example), the layer is said to be dioctahedral; when they occupy all of the cavities (3 cavities out of 3 as in the case of magnesium, for example), the layer is said to be trioctahedral.

The elementary sheets of these clays carry negative charges which are compensated by the presence of exchangeable cations, alkali metal cations such as Li⁺, Na⁺and K⁺, alkaline earth metal cations such as Mg²⁺ and Ca²⁺, and optionally the hydronium ion H₃O⁺. The smectites have charge densities on the sheets which are lower than those of clays of the vermiculite type: approximately 0.66 charge per unit cell as against 1 to 1.4 charge per unit cell for the vermiculites.

The compensating cations are essentially sodium and calcium in the smectites and magnesium and calcium in the vermiculites. From a charge density standpoint, smectites and vermiculites occupy a middle position between talc and pyrophyllite on the one hand, both sheets of which are neutral, and micas on the other, characterized by a high charge density on the sheets (approximately 2 per unit cell) which is generally compensated by K⁺ ions.

The interlayer cations of the smectites and vermiculites can be replaced quite easily by ion exchange by other cations such as, for example, ammonium ions or ions of alkaline earth metals or of tare earth metals.

The swelling properties of the clays depend on a variety of factors, including the charge density and the nature of the compensating cation.

Thus the smectites, whose charge density is lower than that of the vermiculites, have swelling properties which are markedly greater than those of the latter, and therefore constitute a highly interesting class of solids. The repeating distance or basal spacing represents the shortest distance separating two crystallographically identical units situated in two adjacent sheets. As a result of swelling, the basal spacing in the smectites may thus attain values ranging from approximately 1 nm to more than 2 nm.

Among the “swelling” phyllite-type silicates of the smectite type, mention may be made of the following main solids of general formula:

(M₁ⁿ⁺)_{x/n}(M₂)₂^{VI}(M₃)₄^{IV}O₁₀(OH)₂

in which M₁ is the interlayer cation

M₂ is the metal in octahedral position

M₃ is the metal in tetrahedral position

x is the number of charges contributed by the cation M₁

The dioctahedral smectites.

| | |
|---|---|
| montmorillonite | (H, Na, Ca_{1/2})ₓ(MgₓAl₂₋ₓ)^{VI}Si₄^{IV}O₁₀(OH)₂ |
| beidellite | (H, Na, Ca_{1/2})ₓAl₂^{VI}(AlₓSi₄₋ₓ)^{IV}O₁₀(OH)₂ |
| nontrolite | (H, Na, Ca_{1/2} . . .)ₓ(Fe, Al)₂^{VI}(AlₓSi₄₋ₓ)^{IV}O₁₀(OH)₂ |

The trioctahedral smectites

| | |
|---|---|
| hectorite | Naₓ(LiₓMg₃₋ₓ)^{VI}Si₄^{IV}O₁₀(OH)₂ |
| saponite | Naₓ Mg₃^{VI}(AlₓSi₄₋ₓ)^{IV}O₁₀(OH)₂ |
| stevensite | Na₂ₓMg₃₋ₓ^{VI}Si₄^{IV}O₁₀(OH)₂ |

Following saturation adsorption of water or an organic polar solvent in a smectite, the interlayer spacing (between two sheets) is at its maximum. It may attain a value in the region of 1-nm.

These solids are therefore of potential interest in catalysis because their potential specific surface and their potential acidity are high.

According to one preferred embodiment of the invention, the clay which constitutes the catalyst for cyclizing hydrolysis of the aminonitrile to a lactam is a smectite. More preferably, the clay is montmorillonite.

Unfortunately, some clays have the disadvantage of losing their expanded character by heating at 100° C. and, consequently, of failing to conserve the increase in specific surface resulting from their expansion. This is the case in particular with smectites.

Various methods have been described in the prior art for introducing pillars or bridges between the smectite sheets in order to obtain pillared smectites which maintain a high interlayer spacing after having been subjected to a heat treatment. One method, which comprises introducing pillars consisting of oligomers of a metal hydroxide, particularly aluminium hydroxide, was described by Lahav, Shami and Shabtai in Clays and Clay Minerals, Vol. 26 (No. 2). pp. 107-115 (1978) and in French Patent 2,394,324. The formation of pillars consisting of oligomers of mixed silicon and boron hydroxides is described in U.S. Pat. No. 4,248,739. A technique of pillaring smectites by dialysis using hydroxides of aluminium, of chromium, of zirconium and titanium etc. is claimed in Patent EP 0 073 718.

The principle of these methods consists in contacting the clay with a solution containing more or less oligomerized ionic species of the hydroxy-aluminium (in the case of aluminium) type. This operation is generally conducted in a low-concentration solution at a temperature of less than 80° C. and, where possible, in the absence of clouding due to incipient is precipitation of the metal hydroxide. The concentrations of the metal ion and of the clay must be optimized so that sufficient solid pillars are formed and so that the porosity of the clay is not greatly diminished by the insertion of too large an amount of metal oxide.

When the interlayer alkali metal or alkaline earth metal ions are replaced by protons, either directly using a very dilute solution or, preferably, by exchange with an ammonium salt followed by calcination at between 300 and 700° C., the pillared smectites acquire an acidity which, although high, is lower overall than those of the classic zeolites of type Y or mordenite, for example.

According to one preferred embodiment of the invention, the clay used as catalyst for the cyclizing hydrolysis of the aminonitrile to a lactam is pillared.

According to one particular variant of the invention, the catalyst may comprise, besides a clay, one or more other metal compounds, often referred to as dopants, such as, for example, compounds of chromium, titanium, molybdenum, tungsten, iron or zinc. Among these dopants, the compounds of chromium and/or of iron and/or of titanium are considered as being the most advantageous. By weight based on weight of clay, these dopants commonly represent from 0% to 10% and preferably from 0% to 5%.

By metal compound is meant not only the metal element but also the metal ion or any combination comprising the metal element.

The clay according to the invention can be calcined, by a technique which is known to the person skilled in the art.

According to another particular variant of the invention, the catalyst is employed in bead form, in crushed form, as extrudates in the form of hollow or solid cylindrical or multilobate granules or honeycombs or as pellets.

The shaping operation may where appropriate be accomplished with the aid of a binder.

The catalyst may comprise, first, beads of clay obtained from an oil-drop shaping operation (or drop coagulation). Beads of this type may be prepared, for example, by a process similar to that described for the formation of alumina beads in Patents EP-A-0 015 801 or EP-A-0 097 539. Control over the porosity may be achieved in particular, in accordance with the process described in Patent EP-A-0 097 539, by drop coagulation of an aqueous dispersion or suspension of clay.

The beads may also be obtained by the process of agglomeration in a rotating drum or granulator.

The catalyst may also comprise clay extrudates. These can be obtained by blending followed by extrusion of a material based on clay. In these extrudates, the porosity can be controlled by selecting the clay employed and by the conditions under which this clay is prepared or by the conditions under which this clay is blended before extrusion. Thus the clay may be mixed with pore formers during blending. By way of example, the extrudates may be prepared by the process described in U.S. Pat. No. 3,856,708.

Similarly, beads with controlled porosity may be obtained by adding pore former and carrying out agglomeration in a rotating pan or granulator or by the oil-drop process.

Thus, as will be demonstrated below, the cyclizing hydrolysis reaction can be conducted with a minimum of side reactions, with good selectivity of the process for lactam and hence a high purity of the crude product obtained.

The aminonitriles which can be cyclized by the process of the invention are, advantageously, aliphatic ω-aminonitriles such as ω-aminovaleronitrile, ω-aminocapronitrile, ω-aminooctanonitrile, ω-aminononanonitrile, ω-aminodecanonitrile, ω-aminoundecanonitrile, ω-aminododecanonitrile and methylaminovaleronitrile.

The preferred and most important-compound is 6-aminocapronitrile, which leads to ε-caprolactam. The latter compound is the monomer of polyamide 6, which is used to manufacture various articles such as mouldings, yarns, fibres, filaments, cables or films.

The ε-caprolactam produced by the cyclizing hydrolysis reaction is advantageously purified by various known purification processes, such as distillation, crystallization in a solvent medium or a melt phase, treatment on resin, treatment by an oxidizing agent, and/or hydrogenation. These various steps may be combined in part or in whole in different orders and in accordance with the degree of purity of the ε-caprolactam produced.

The cyclizing hydrolysis reaction requires the presence of water. The molar ratio between the water and the aminonitrile that are employed is normally between 0.5 and 50 and preferably between 1 and 20. The upper value of this ratio is not critical for the invention, but higher ratios are of virtually no interest economically.

According to one particular embodiment of the invention, the cyclizing hydrolysis reaction is implemented in vapour phase. Accordingly, the reactants are maintained in the vapour state in the reactor, which is charged with a defined quantity of catalyst. The aminonitrile and the water may be employed in the form of the mixtures in the vapour state, or may be introduced separately into the reactor. It is possible to vaporize the reactants beforehand, which then circulate within a mixing chamber.

According to another particular embodiment of the invention, the cyclizing hydrolysis reaction is implemented in liquid phase. Accordingly, the aminonitrile and water-reactants are employed in the liquid state under pressure, optionally in the presence of a solvent.

The free volume of the reactor may be occupied by an inert solid such as, for example, quartz in order to promote the vaporization and dispersion of the reactants.

It is possible without disadvantage to use any inert gas as carrier, such as nitrogen, helium or argon.

The temperature at which the vapour phase process is implemented is advantageously sufficient for the reactants to be properly in the vapour state. This temperature is generally between 200° C. and 450° C. and preferably between 250° C. and 400° C.

In the case of a liquid phase process, the reaction is conducted under pressure.

The contact time between the aminonitrile and the catalyst is not critical. It may vary depending on the apparatus used, in particular. This contact time is preferably between 0.5 and 200 seconds and more preferably still between 1 and 100 seconds.

The pressure is not a parameter critical to the process. Thus it is possible to operate under pressures of from 10⁻³ bar to 200 bar. Preferably, the process will be implemented under a pressure of from 0.1 to 20 bar. In the case of a hydrolysis conducted in vapour phase, this pressure is advantageously between 10⁻³ bar and 3 bar.

The use of a solvent that is inert under the reaction conditions, such as, for example, an alkane, a cycloalkane, an aromatic hydrocarbon or a halogenated form of one of these above hydrocarbons, and thus the presence of a liquid phase in the reaction flow, is not ruled out.

Examples will now be given, solely by way of indication and for the purpose of illustrating the invention.

### EXAMPLE

A 20 ml cylindrical Pyrex glass reactor with a fixed bed, arranged vertically and equipped with heating means and a temperature probe, openings for the entry and exit of the gaseous flows and for the entry and exit of the liquid flows, and a system for injecting the reactants, is charged in succession with 10 g of quartz beads, 2 g of pillared montmorillonite from Fluka®, ref. 69907, in the form of a 0.8 to 1.25 micrometre powder, mixed or not mixed with 2 g of quartz beads, and a further 10 g of quartz beads.

The reactor thus charged is heated at 300° C. under a stream of nitrogen (with a flow rate of 5.2 litres/hour) for 2 hours.

By means of a pump, a mixture of 6-amino-capronitrile (ACN) and water (molar ratio water/ACN of 4.1) is then injected. The rate of injection of the mixture is 4.34 ml/h.

At the outlet of the reactor, the vapours are condensed in a glass trap at ambient temperature over a period of 2 hours: the reaction product is thus recovered in two successive cuts of 1 h.

The final reaction mixture (the second fraction withdrawn) is assayed by gas chromatography in order- to determine, in particular, the caprolactam concentration.

The parameters determined are the degree of conversion (DC) of the aminocapronitrile, the selectivity (S) for caprolactam (CPL) relative to the aminocapronitrile converted, the activity of the catalyst over 2 hours of reaction, measured in grams of caprolactam formed per gram of catalyst per hour.

The following consistent performance figures are obtained for a reaction period of 50 hours:

| | |
|---|---|
| DC of the ACN: | 19.6% |
| S for CPL: | 92.8% |
| activity: | 6.25 g/g · h |

1. Process for cyclizing hydrolysis of an aminonitrile compound to a lactam by reaction of an aminonitrile of general formula (I):
N≡C—R—NH₂ (I)
in which r represents a substituted or unsubstituted, linear or branched, saturated or unsaturated aliphatic, cycloaliphatic or arylaliphatic radical containing from 1 to 12 carbon atoms with water in the presence of a solid catalyst, characterized in that the catalyst is a clay.

2. Process according to claim 1, characterized in that the clay is selected from kaolins, serpentines, smectites or montmorillonites, illites or micas, glauconites, chlorites or vermiculites, attapulgites or sepiolites, mixed-layer clays, allophanes or imogolites, and clays with a high alumina content.

3. Process according to claim 1 or 2, characterized in that the clay is a montmorillonite.

4. Process according to one of the preceding claims, characterized in that the clay is pillared.

5. Process according to one of the preceding claims, characterized in that the catalyst comprises one or more dopants.

6. Process according to one of the preceding claims, characterized in that the catalyst is employed in bead form, in crushed form, as extrudates in the form of hollow-or solid cylindrical or multilobate granules or honeycombs, or as pellets.

7. Process according to one of the preceding claims, characterized in that the aminonitrile of the formula (I) is 6-aminocapronitrile.

8. Process according to one of the preceding claims, characterized in that the molar ratio between the water and the aminonitrile that are employed is between 0.5 and 50.

9. Process according to claim 8, characterized in that the molar ratio between the water and the aminonitrile that are employed is between 1 and 20.

10. Process according to one of the preceding claims, characterized in that the cyclizing hydrolysis reaction is implemented in vapour phase.

11. Process according to one of claims 1 to 9, characterized in that the cyclizing hydrolysis reaction is implemented in liquid phase.

12. Process according to one of claims 1 to 10, characterized in that the temperature at which the step of cyclizing hydrolysis is implemented is between 200° C. and 450° C.
